(19) United States
(12) Patent Application Publication
(10) Pub. No.: US 2005/0267347 A1
(43) Pub. Date: Dec. 1, 2005

(54) Deep brain stimulation

(76) Inventor: Doran Oster, Gainesville, FL (US)

Correspondence Address: Alison L. McCarthy, Esq.;Mintz Levin Cohn Ferris Glovsky & Popeo, P.C., One Financial Center, Boston, MA, 02111, US

(21) Appl. No.: 11/121,719

(22) Filed: May 4, 2005

Related U.S. Application Data
(60) Provisional application No. 60/567,863, filed on May 4, 2004.

(51) Int. Cl. A61B 5/04; A61B 5/05

(52) U.S. Cl. 600/378; 600/407

(57) ABSTRACT

A probe used in deep brain stimulation includes a cannula comprising an elongated housing defining an internal aperture and having a base portion with a notch, the housing having a longitudinal axis, and an electrode configured to be inserted through the aperture of the cannula. The electrode and notch are configured such that the electrode will contact the notch when inserted in the cannula and be directed out of the cannula at a non-zero angle relative to the longitudinal axis of the housing.

### RELATED APPLICATIONS

This application claims priority to U.S. Provisional Application Ser. No. 60/567,863, filed on May 4, 2004 and entitled, “Deep Brain Stimulation,” which is herein incorporated by reference.

### BACKGROUND OF THE INVENTION

Many neurological conditions, brain diseases and malfunctions are manifested by changes in the electrical and chemical behavior of groups of cells. For example, some types of tremors, including those suffered by Parkinson's patients, are caused by a small group of deep brain cells that discharge at an uncharacteristic rate. Often these symptoms can be reduced or eliminated if these cells are treated by placing one or more neurostimulators within the proper deep brain structure. These neurostimulators apply a voltage to the immediate neighborhood of cells such that they no longer participate in overall brain function. In many cases, this form of treatment has been shown to benefit the patient while avoiding the side effects inherent in drug therapy.

This procedure is a complex one with very little tolerance for error. The most important factor affecting the success of treatment is accuracy of neurostimulator placement. The targets cannot be detected with imaging methods such as CT, MRI, and PT-scans. Instead, surgeons insert probes tipped with thin electrodes into the brain and observe and record the electrical characteristics of individual cells visually with oscilloscopes and audibly by converting the electrical signals to sound. By evaluating these recordings, neurosurgeons refine their mapping of the patient's brain with the degree of resolution necessary to isolate the target.

Obtaining and evaluating electrode recordings are the most invasive, time consuming, and error prone components of deep brain stimulation treatment. It is invasive because surgeons are forced to move the electrode in “straight-line” trajectories originating from a point outside the brain. Using typical techniques, when the doctors need to collect recordings at new locations, they completely withdraw the electrode and reinsert it on a new trajectory into the deep brain. Typically four or five such insertions are required, causing a degree of brain tissue damage in the process. This process is also the most time consuming as each trajectory takes time to be accurately oriented and the electrodes must move slowly through the brain matter. Finally, the recordings are often noisy or ambiguous requiring a highly skilled and experienced neurologist/physiologist to translate the recordings into a reliable brain map. There are currently no automated tools to reliably interpret these signals and few neurologists capable of performing this specialized task.

### SUMMARY OF THE INVENTION

The innovations described herein are directed to making a deep brain stimulation procedure substantially less invasive, less time consuming, and more reliable while making the procedure more feasible for institutions other than large and specialized institutions.

The invention provides an improved method for inserting locational probes into the brain, recording the data produced by the locational probes, interpreting and storing the collected data, and using that data to guide the probes in ways that reduce risks to the patient and reduce the time in surgery. The invention preferably employs a semi-microelectrode that creates a 3-dimensional map of a portion of the brain and a digital signal processing system that discerns the difference between misfiring cells and normal cells sufficiently to allow at least partial automation of probe placement in deep brain stimulation. The invention provides a suite of tools for use by a surgeon to analyze data detected during deep brain stimulation.

Embodiments of the invention use a computer to control the translation of the locational probe rather than hand controls. In addition, electronic signals are recorded into a computer's memory in real time.

In general, in an aspect, the invention provides a probe used in deep brain stimulation. The probe includes a cannula comprising an elongated housing defining an internal aperture and having a base portion with a notch, the housing having a longitudinal axis, and an electrode configured to be inserted through the aperture of the cannula. The electrode and notch are configured such that the electrode will contact the notch when inserted in the cannula and be directed out of the cannula at a non-zero angle relative to the longitudinal axis of the housing.

Implementations of the invention may include one or more of the following features. The electrode can comprise a semi-microelectrode. The electrode may comprise one of spring-tempered stainless steel or spring-tempered nickel-titanium. The electrode can be directed along at least one of an angle of about 25 degrees, about 30 degrees, about 45 degrees or about 90 degrees relative to the longitudinal axis of the cannula. The notch can be configured to directed the electrode at a predetermined angle with respect to the longitudinal axis of the cannula.

In general, in another aspect, the invention provides a method of mapping a 3-dimensional area of the brain using a semi-microelectrode probe. The method includes inserting a sheath into the brain along a straight-line trajectory to position an aperture of the sheath at a first rotational position and depth, advancing a semi-microelectrode into the sheath to at least the predetermined depth of the sheath, directing the semi-microelectrode to extend at an angle in a direction away from the straight-line trajectory of the sheath to a first location, and collecting electrical data from brain tissue in an area proximal to the first location.

Implementations of the invention may include one or more of the following features. The method may further include withdrawing the semi-microelectrode into the sheath, rotating the sheath such that the aperture of the sheath is located at a second rotational position, re-advancing the semi-microelectrode out of the sheath, directing the semi-microelectrode to extend at the angle in a direction away from the straight-line trajectory of the sheath to a second location, and collecting data in an area proximal to the second location. The method may also include adjusting the depth of the sheath to a second depth. The method may also include adjusting a rotational position of the sheath and a position of the semi-microelectrode relative to the sheath to collect data along a 3-dimensional conical area radiating from a base of the sheath. The method may also include storing the data collected, classifying tissue from the collected data, and mapping an area of the brain according to the data collected and the classified tissue.

In general, in another aspect, the invention provides a system for use in a deep brain stimulation procedure. The system includes a memory unit configured to store data associated with a portion of a brain collected during a deep brain stimulation process, and a processor configured to cause display of a brain/probe image including at least one probe image and a 3-dimensional brain image, recording of electrical data detected by a deep brain stimulation probe in association with locations in the brain producing the detected data, and playing of a particular sound corresponding to the recorded data associated with a selected portion of the brain in the 3-dimensional brain image.

Implementations of the invention may include one or more of the following features. The processor can be further configured to display a coordinates table having coordinates of the portion of the brain/probe image at which the probe image is located. An entry in the coordinates table can be selectable to cause playback of the particular sound. The system can include a speaker.

Various aspects of the invention may provide one or more of the following capabilities. Deep brain stimulation (DBS) can be performed in a quicker and less costly manner than the current state of the art. The surgery time during which the brain is exposed is reduced, decreasing the chances of infections and other complications. Patient discomfort and stress can be reduced during DBS. The chances of damaging large veins and arteries in DBS can be reduced. The chances of accidentally damaging specified cells or nerves during DBS can be reduced. Specific cells, for example, the optic nerve for the purpose of implanting prosthesis, may be found, possibly without damaging these cells. The chances of correctly finding the target area for the neurostimulator and the success rate of DBS can be improved. DBS data can be analyzed remotely. A surgeon determining the best position of the antenna for DBS may be located remotely from the operating room. For example, the locational probes could be inserted in a patient in Europe while the data is analyzed and the target area is determined by a group of surgeons in the US who specialize in this procedure. Specialists, preferably with significant experience, can be used despite their location to help improve DBS results. The success of DBS can be correlated with data produced months after an operation. Improvements in the DBS techniques can be accelerated compared to the current pace of improvements. Data from a databank can be used for training new surgeons. DBS can be made available to a larger population of patients. Data from a databank can be used to improve an algorithm controlling the translation of DBS probes, e.g., by adjusting the sampling frequency and/or rate of movement of the probe(s) based upon the region of the brain in which the locational probe(s) is(are) disposed as indicated by the signals detected by the probe(s). DBS functions that currently require a specialized surgeon in the operating room can be automated. DBS can be extended to new applications, such as treating epilepsy, depression, inserting sensory prosthesis, etc. A map of a normal brain can be deduced. DBS signals detected at different times could be played for comparison. Identification and classification of brain cells may be improved. Brain location of target cells, e.g., that may be misfiring, can be predicted. Chances of infection can be reduced. DBS treatments may be more feasible at smaller institutions. The margin for error over current DBS techniques can be increased.

These and other capabilities of the invention, along with the invention itself, will be more fully understood after a review of the following figures, detailed description, and claims.

### BRIEF DESCRIPTION OF THE FIGURES

FIG. 1 is a simplified diagram of a prior art setup for performing deep brain stimulation.

FIG. 2 is a perspective view of an electrode used in deep brain stimulation according to the invention.

FIG. 3 is a perspective view of a cannula that accepts an electrode used in deep brain stimulation according to the invention.

FIG. 4 is a simplified diagram of a setup for performing deep brain stimulation according to the invention.

FIG. 5 is a screen shot of an application interface according to the invention.

FIG. 6 is a screen shot of a 3-D brain image according to the invention.

FIG. 7 is a table of coordinates for brain cell identified according to the invention.

### DETAILED DESCRIPTION OF PREFERRED EMBODIMENTS

Embodiments of the invention are directed to techniques for deep brain stimulation. Embodiments of the invention provide electrodes for mapping 3-dimensional areas of the brain. For example, embodiments of the invention may use a splaying electrode or an electrode that can be inserted and re-inserted into a cannula positioned in the brain. Embodiments of the invention allow at least partial automation of placement of a probe in deep brain stimulation. Embodiments of the invention include methods, using digital signal processing techniques, of electronically discerning the difference between normal nerve firings and improper nerve firings. Electronic differentiation can be used in a system of motor-driven, computer-controlled probe or probes that could be driven down at a constant rate by a motor. In embodiments of the invention, a computer is used to control the translation of a locational probe rather than hand controls used by a surgeon. Other embodiments are within the scope of the invention.

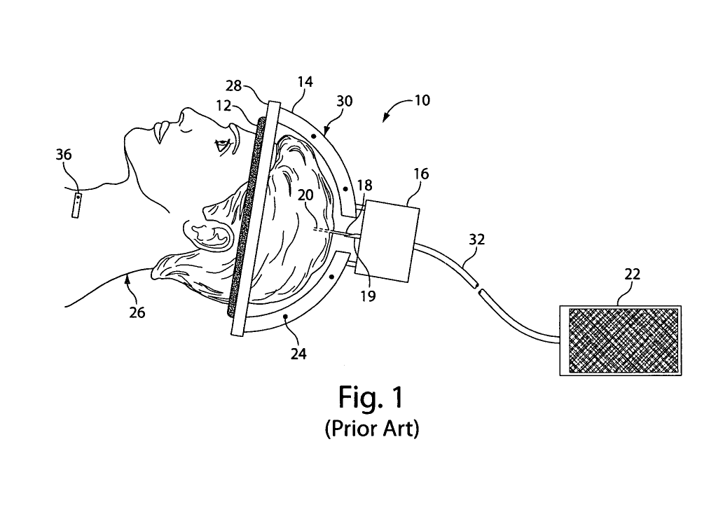

In one technique of Deep Brain Stimulation (DBS), locational probes are used to locate a group of misfiring cells and aid in the positioning of a neurostimulator. Referring to FIG. 1, a neurostimulator 10 is positioned on a patient upon location of a target. The neurostimulator 10 includes a halo ring 12, a semicircular (hemispherical) ring 14, a servomotor 16, a probe 18 including a cannula 19 and an electrode 20, and a speaker 22. The halo ring 12 includes reference marks 24. The probe 18 is configured to be inserted into brain matter for DBS. The reference marks 24 correlate with a target area of the brain of a patient 26.

The halo ring 12 encircles the head of the patient 26 and is fixed to the skull. At the start of an operation, the halo ring 12 is fixed to the operating table on which the patient 26 rests, substantially preventing movement of the patient's head. A hole is opened in the patient's scalp and skull to create a portal to the brain in a predetermined position. A second flat ring 28 is mated to the initial ring 12. The second ring 28 contains the hemispherical ring member 14 that forms an arc 30 over the opening in the skull. The servomotor 16 is attached to the ring 14. The motor 16 is oriented so that the center axis of the motor 16 aligns along a path that intersects the location of the target region. The probe 18 is inserted into a hole in the motor 16.

The translation of the probe 18 along the vertical axis is controlled via an encoded data wheel. The electrode 20 at the end of the probe 18 monitors and transmits the electrical pulses of individual cells through a lead 32 connected to an audio amplifier. The pulse is amplified and sent to speakers 22 located in the operating theater. The pulse may also be sent to oscilloscopes located in the operating room/theater. The various types of brain cells produce characteristic sounds. The surgeon interprets the sounds to identify the types of cells along the path of the probe 18 and the information is transcribed into a spreadsheet that maps the position of the electrode 20 and the type of cell. Typically, it takes about 50 minutes to align the motor 16 and record the data from the electrode 20. The process is repeated, e.g., as many as six times, with the probe 18 aligned to different positions around the expected target until the surgeon has enough data to determine/deduce the position of the target cells.

When the target area has been determined, the electrode 20 is inserted into the target area with electrical leads attached. Typically, the electrode 20 is a microelectrode. The microelectrode 20 is moved in straight-line trajectories from a fixed starting position. The electrode 20 isolates signals from single neurons. The leads are routed through the brain, out of the opening in the skull and under the patient's skin to a controller 36 embedded near the collarbone. The controller 36 powers and controls an electronic signal emitted by the electrode 20.

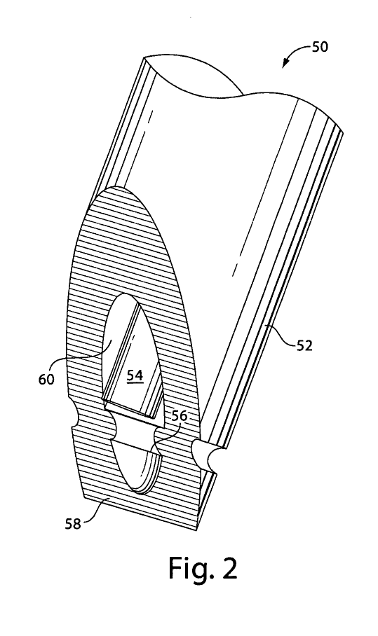

Referring to FIG. 2, in a preferred embodiment of the invention, a DBS probe 50 includes a cannula 52 and an electrode 54. The cannula 52 is a tube acting as a guide or sheath for the electrode 54. The cannula 52 includes a raised notch 56 at the base 58 of the cannula 52. The tip of the cannula 52 is angled. The electrode bends at an angle from the primary trajectory of the cannula 52 through the tip of the cannula 52. The electrode 54 is inserted through the cannula 52 and enters an area in the brain through a center aperture 60 of the cannula 52.

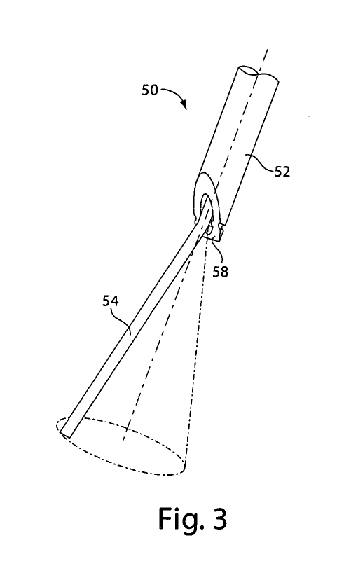

As discussed, the cannula 52 is positioned in the brain for determining a target area of the brain to be stimulated. The electrode 54 is inserted through the cannula 52 and contacts the notch 56. Referring also to FIG. 3, the notch 56 causes the electrode 54 to bend at an angle, i.e., the electrode 54 does not extend in the same straight-line trajectory as the cannula 52. The notch 56 determines the angle at which the electrode 54 extends when exiting the cannula 52. For example, the notch 56 can cause the electrode to extend at about angles of 20-degrees, 30-degrees, 45-degrees, or 90-degrees, or other angles. The offshoot direction of the electrode 54 can be adjusted by withdrawing the electrode 54 into the cannula 52 and rotating the cannula 52. The electrode 54 can be re-advanced through the cannula 52. A 3-dimensional area can be mapped by the insertion and adjustment of the electrode 54 into the cannula 52 with the cannula 52 rotated to different positions. For example, by rotating the cannula 52 and adjusting the electrode 54, a 3-dimensional conical area can be mapped.

The cannula 52, in addition to being rotated, can be adjusted in the translational direction, i.e., deeper or less deep into the brain. By adjusting the depth, the electrode 54 maps a different 3-dimensional area of the brain at different depths using the same cannula 52. A series of conical areas radiating from the base of the cannula 52 can be mapped as the cannula 52 is adjusted in depth and rotation and the electrode 54 is inserted one or more times.

The electrode 54 can be a microelectrode, or, preferably, a semi-microelectrode. The electrode 54 can be a flexible electrode made of a spring-like material, such as spring-tempered stainless steel or nickel-titanium. Various materials such as a material that is preferably flexible, has a strong restoration force, has an appropriate electrical impedance, and achieves FDA approval, can be used for the electrode 54. Employing a flexible material for the electrode 54 allows surgical teams to more delicately explore and map deep brain structures, although other less flexible electrodes can be used.

A flexible electrode 54, such as a semi-microelectrode, may have a higher impedance than a microelectrode. Higher impedance electrodes obtain signals in the brain from a population of neurons, rather than a single neuron. Using a higher-impedance electrode provides a much higher signal-to-noise ratio than using a low impedance electrode, for estimation of brain structure borders.

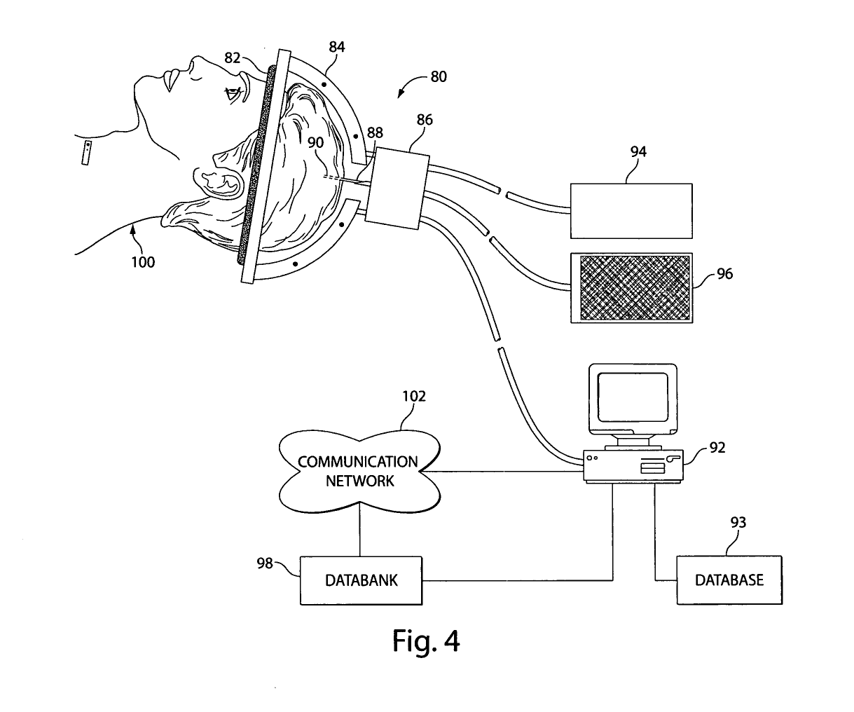

The electrodes used for deep brain stimulation (both microelectrodes and semi-microelectrodes) are guided into the brain to acquire data. Referring to FIG. 4, a deep brain stimulation data acquisition system for guiding the electrode, acquiring data, interpreting the collected data, and estimating a target location includes a neurostimulator 80, a halo ring 82, a semi-circular or hemispherical ring 84, a servomotor 86, a locational probe 88 having an electrode 90, a computer 92, a database 93, a communication network 102, an oscilloscope 94, a speaker 96 and a databank 98. The halo ring 82 and the semi-circular ring 84 are positioned for stabilizing a patient 100 and targeting a region of the patient's brain. The servomotor 86 drives the locational probe 88. The computer 92 is connected to the servomotor 86.

The computer 92 is programmed to guide the electrode 90 into the proper position in the brain. The computer 92 is configured to receive signals detected by the electrode 90 and to drive the rotational angle and depth of the probe 88. The computer 92 automatically causes translation of the locational probe 88 along its path within preset limits. The computer 92 translates the probe 88 in steps. The size of the steps is controlled by a set of rules that reduces the time of the procedure while collecting pertinent data. The procedure during which data is collected can be repeated in several locations using a single probe 88. Alternatively, several probes 88 can be used substantially simultaneously to reduce the time to complete the data collection.

The computer 92 can be programmed to control movement of the probe 88 in a specified manner. For example, the computer 92 can be programmed to stop if the probe 88 approaches a vein. The computer 92 can be programmed with coordinates of veins, or other objects to avoid, which are determined from MRI images, prior DBS procedures, etc. The sound of the patient's pulse or the pressure wave caused by blood moving through vessels may be used to identify objects such as blood vessels to avoid. The computer 92 can be programmed to stop if the probe 88 closely approaches or touches unexpected or protected cells, such as optic nerve cells. The surgeon preferably can override the computer 92 at any point during the procedure.

The computer 92 can triangulate the target area. For example, the computer 92 can search along a path of each probe for the sound of target cells. If the computer 92 finds target cells on more than one path, then the computer 92 determines the location of the target using the combined data, e.g., to determine a location between the paths and/or locations of detected target cells as the target region. The computer 92 may detect target cells and/or determine the location of target cells if the electrodes are close to, but not actually touching, the target cells.

The computer 92 can predict an optimum path to be followed by a probe 88 to locate the target area, e.g., using the data collected from the probe(s) 88 previously or currently inserted into the patient 100. For example, if the computer 92 detects a region that showed no correlation to target cells, then the next probe 88 could be spaced further away than originally planned to increase the chances of finding the target and possibly decreasing the number of probe insertions used to find the target cells. The computer 92 uses information from the databank 98 to predict the path for the next probe 88. If target cells are located, future probes 88 can be placed in order to find the boundaries of the target area. Once the probes 88 have been translated the desired amount, e.g., the entire length of each path of the probes 88, the opening in the skull can be temporarily closed while the data is analyzed.

For use in determining how to guide the electrode 90, the computer 92 and the database 93 are configured to store, organize and transmit information used by the surgeon during DBS treatment. The computer 92 is connected to the database 93 and the databank 98 directly and/or indirectly through the communications network 102, such as the Internet. The computer 92 can be configured to send data from the electrode 90 over the network 102 to remote locations, e.g., for analysis by surgeons at locations remote from an operating room where the patient 100 is disposed. The computer 92 can also be configured to send data from the electrode 90 to the database 93 and/or the databank 98 for storage, and to a local memory of the computer 92 for storage, manipulation by a processor of the computer 92, and display on a screen of the computer 92.

The database 93 stores a patient's medical imaging (MR, CT, etc.), neurological diagnosis and evaluations, and results of previous DBS treatments, if any. In addition, recordings made from the electrode 90 and collected during DBS surgery are added to the database 93. Preferably, the recordings are added to the database 93 in real time. Collectively, the information stored in the database is used by the computer 92 to guide the probe 88 during a procedure.

The computer 92 focuses on the detection of brain structure borders as a method of identifying target locations. The recordings (either microelectrode or semi-microelectrode) are used to decipher the locations of brain structures, in addition to individual neurons. The computer 92 is configured to create 3-D models of the brain structures using a standard atlas, such as the Shaltenbrand atlas. From the 3-D reconstruction, a target location is estimated and a rapid search pattern is plotted to confirm the target estimate.

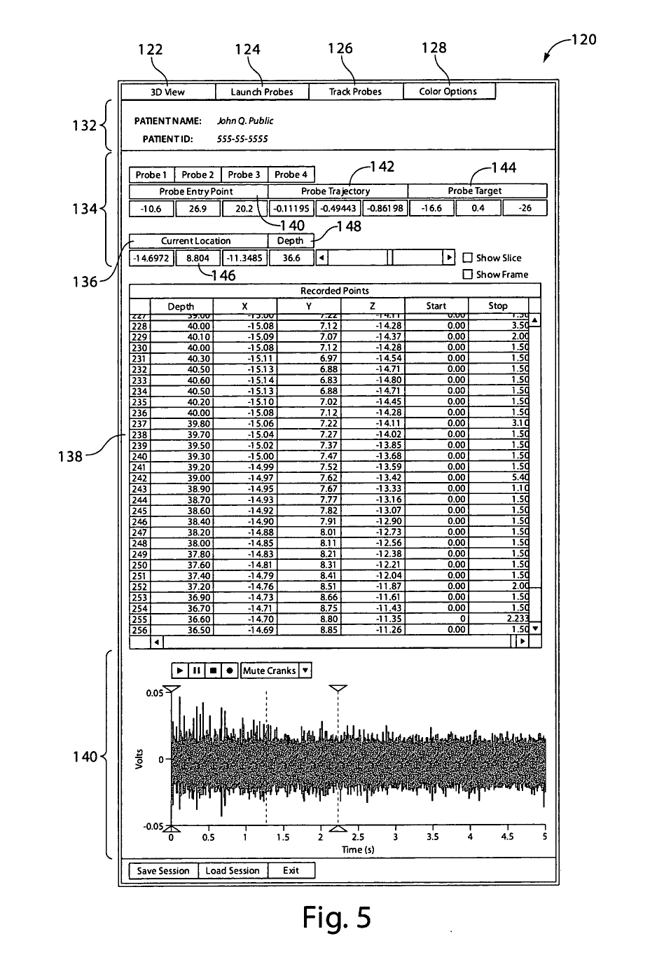

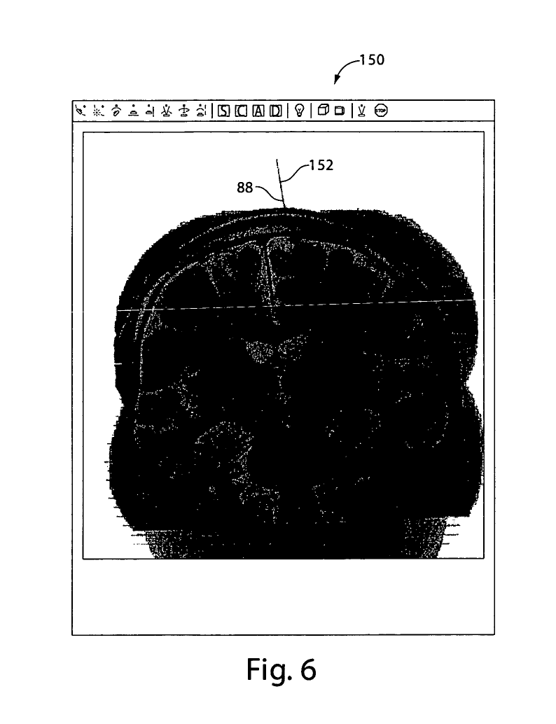

Referring also to FIGS. 5 and 6, the computer 92 is configured to run an application that pools the stored information and presents it in an integrated environment/format. An application interface 120 includes a 3-D viewer option 122, a launch probes option 124, a track probes option 126 and a color option 128. The 3-D viewer option 122 allows a user to view a 3-D image (shown in FIG. 6) of all or a portion of the brain. The launch probes option 124 coordinates the insertion and control of a probe for real-time DBS. The track probes option 126 allows real-time tracking of the probe 88. The color option 128 allows the assignment and adjustment of color tracking of images of the brain.

In FIG. 5, the track probes option 126 is selected. With the track probes option 126 selected, the interface 120 includes a patient information field 132, trajectory information 134, a current probe locator 136, microelectrode recordings 138 and playback controls 140. The trajectory information 134 provides probe entry point information 140, a probe trajectory 142, a probe target 144, a coordinate reading 146 for current location of a probe and a depth reading 148. The microelectrode recordings 138 present data from identified brain cells that have been converted to audio, taken previously.

The application interface 120 allows the surgeon, through selection of the 3-D viewer option 122, to display a 3-D image 150 of the patient's brain (e.g., from a previous MRI or similar imaging method) with an image 152 of the probe 88 superimposed in its starting position. The 3-D image can present many probes 88 superimposed in their respective starting positions simultaneously. The computer 92 is configured to analyze the signals recorded at each position in which the probe 88 was inserted and determine the type of cell at the various positions along the path. For example, a cell 154 has been identified at the end of the probe 88. Different cell types are displayed with easily discernable attributes, such as with different colors. A 3-D map of the brain in the probed area is created based on the identification of the cell types and locations. The computer 92 is configured to devise an electrode search pattern that increases the chances of successful target location while minimizing the chances of puncturing critical brain structures. The surgeon can confirm or reject the application's automated solutions.

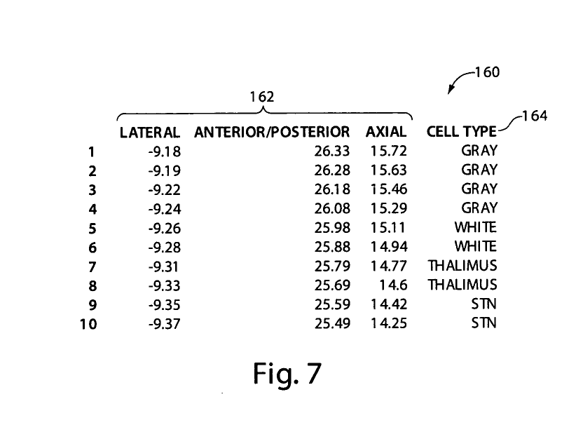

Images of the probes 152 in position in the brain can be selected and information about the current status of the probe 88 presented. Referring also to FIG. 7, each probe 88 has a corresponding 2-D table 160 that lists the 3-D coordinates 162 of each data collection point and the name of the cell type 164. The table 160 can further include a voltage vs. time representation of the pulse. The image of the probe 152 can be made “active” and highlighted in the image 150 by clicking on its image 152 or selecting the corresponding table 160 entry. Putting the cursor on a row in a table 160 causes the image 152 of the probe to move to the corresponding position in the brain image 150 and causes the computer 92 to play back the corresponding recorded sounds. Sounds recorded at drastically disparate times during DBS could be replayed consecutively for more accurate comparison/diagnosis. An active probe can be dragged/moved to various positions along its virtual path, e.g., with a mouse or other computer peripheral device, and the corresponding recorded sound played back.

The computer 92 is configured to interpret and analyze the data collected. A surgeon can interact with the application interface 120. For example, the application interface 120 allows the surgeon an opportunity to play back and analyze microelectrode recordings using the data in the microelectrode recordings 138 and the playback controls 140. Prior probe recordings can be selected for playback. The corresponding position in the table 138 or the table 160 is highlighted and the corresponding recorded sounds are reproduced. The surgeon can toggle between the various probes to compare the sounds at various positions by clicking on the various probe entries, and/or moving probe images, and/or selecting different probe images, e.g., one at a time. Two or more probes can be activated simultaneously. Different types of cells can be distinguished and identified, as well as interfaces between regions of different cell types, and regions of necrosis, etc. The computer 92 analyzes the data and indicates the most likely location of the target cells with a symbol, such as an outline, or an attribute such as a color, or both. The computer 92 displays the 3-D coordinates of the target area.

Information stored in the database 93 is used locally by a surgeon or other provider before, during and/or after a DBS procedure. The centralized databank 98 stores a large number of data sets from previous DBS surgeries or other resources. Data collected during DBS treatment and stored in the database 93 is uploaded to the centralized databank 98 of DBS cases. The uploaded data includes medical imaging (relevant MRI, CT, etc.), neurological diagnoses, and real-time electrode recordings. The information collected can be stripped of identifiers and is HIPPA compliant. The centralized databank 98 correlates the new data with previously collected data and uses the information to plan and guide a surgery. The databank 98 provides a mapping of the brain using microelectrodes and/or semi-microelectrodes as described in FIGS. 2 and 3. A full mapping of the brain using the signals provided by semi-microelectrodes facilitates automated DBS treatment.

In embodiments of the invention, external stimuli may be introduced to help locate/identify cells. For example, to locate the optic nerve, a strobe light can be shone into the patient's eye and the optic nerve located/identified by detecting signals in the brain corresponding to the stimulus. Similar techniques can be applied for other cells, e.g., by applying audio stimuli to identify auditory cells in the patient's brain, or by manipulation of limbs or muscles to identify certain related cells or nerves in the patient's brain, or by the application of touch, manipulative pressure or compression to various parts of the patient's body to identify related cells or nerves in the patient's brain, or by introducing smells to identify smell-related cells or nerves in the patient's brain. Still other stimuli may be used.

A map of a normal brain can be deduced by analyzing the data from many brains that are partially normal and partially diseased. The data library compiled by this method can provide information for a complete map of the human brain. The map of the brain can be used to automatically drive the probes. If detected signals indicate that a probe is in one section of the brain and it is known that the target is in a different section, then the motor can be controlled to move the probe faster and/or a sampling rate of the detected signals can be slowed (i.e., sample less frequently). Preferably, a large sample of signals from a large number of patients are stored in the data bank. These data can be used to determine signal characteristics of cells and associate characteristics with cell types for use in differentiating cell types from detected signals during DBS.

Other embodiments than shown or described are within the scope of the invention. For example, the cannula can accept more than one electrode. One or more than one electrode can extend along the trajectory of the cannula, while another electrode can contact the notch and be directed in an offshoot direction other than the straight-line trajectory. Still further, more than one notch can be positioned at the base of the cannula such that electrodes are directed in more than one offshoot direction during a single procedure. An electrode can extend along the trajectory of the cannula, while more than one electrodes extend at offshoot angles for the collection of data. A cannula can be configured to hold N electrodes having N offshoot directions when inserted into the cannula. The system can also use different configurations for mounting the motor to the patient, such as the MicroTargeting Platform tripod made by FHC of Maine. A 3-D mapping of the brain can be accomplished using a microelectrode, a semi-microelectrode or an electrode inserted into the brain. The database 93 and the databank 98 can be combined to store data collected during deep brain stimulation, rather than storing the information in a database and uploading it into a databank.

Other embodiments are within the scope and spirit of the appended claims. For example, due to the nature of software, functions described above can be implemented using software, hardware, firmware, hardwiring, or combinations of any of these. Features implementing functions may also be physically located at various positions, including being distributed such that portions of functions are implemented at different physical locations.

1. A probe used in deep brain stimulation, the probe comprising:
a cannula comprising an elongated housing defining an internal aperture and having a base portion with a notch, the housing having a longitudinal axis; and
an electrode configured to be inserted through the aperture of the cannula;
wherein the electrode and notch are configured such that the electrode will contact the notch when inserted in the cannula and be directed out of the cannula at a non-zero angle relative to the longitudinal axis of the housing.

2. The probe of claim 1, wherein the electrode comprises a semi-microelectrode.

3. The probe of claim 1, wherein the electrode comprises one of spring-tempered stainless steel or spring-tempered nickel-titanium.

4. The probe of claim 1, wherein the electrode is directed along at least one of an angle of about 25 degrees, about 30 degrees, about 45 degrees or about 90 degrees relative to the longitudinal axis of the cannula.

5. The probe of the claim 1, wherein the notch is configured to directed the electrode at a predetermined angle with respect to the longitudinal axis of the cannula.

6. A method of mapping a 3-dimensional area of the brain using a semi-microelectrode probe, the method comprising:
inserting a sheath into the brain along a straight-line trajectory to position an aperture of the sheath at a first rotational position and depth;
advancing a semi-microelectrode into the sheath to at least the predetermined depth of the sheath;
directing the semi-microelectrode to extend at an angle in a direction away from the straight-line trajectory of the sheath to a first location; and
collecting electrical data from brain tissue in an area proximal to the first location.

7. The method of claim 6, further comprising:
withdrawing the semi-microelectrode into the sheath;
rotating the sheath such that the aperture of the sheath is located at a second rotational position;
re-advancing the semi-microelectrode out of the sheath;
directing the semi-microelectrode to extend at the angle in a direction away from the straight-line trajectory of the sheath to a second location; and
collecting data in an area proximal to the second location.

8. The method of claim 7, further comprising adjusting the depth of the sheath to a second depth.

9. The method of claim 6, further comprising adjusting a rotational position of the sheath and a position of the semi-microelectrode relative to the sheath to collect data along a 3-dimensional conical area radiating from a base of the sheath.

10. The method of claim 6, further comprising:
storing the data collected;
classifying tissue from the collected data; and
mapping an area of the brain according to the data collected and the classified tissue.

11. A system for use in a deep brain stimulation procedure, the computer comprising:
a memory unit configured to store data associated with a portion of a brain collected during a deep brain stimulation process; and
a processor configured to cause:
display of a brain/probe image including at least one probe image and a 3-dimensional brain image;
recording of electrical data detected by a deep brain stimulation probe in association with locations in the brain producing the detected data; and
playing of a particular sound corresponding to the recorded data associated with a selected portion of the brain in the 3-dimensional brain image.

12. The system of claim 11, wherein the processor is further configured to display a coordinates table having coordinates of the portion of the brain/probe image at which the probe image is located.

13. The system of claim 12, wherein an entry in the coordinates table is selectable to cause playback of the particular sound.

14. The system of claim 11, wherein the system includes a speaker.
